(19) United States
(12) Patent Application Publication
(10) Pub. No.: US 2009/0041687 A1
(43) Pub. Date: Feb. 12, 2009

(54) Use of opioid receptor antagonists

(76) Inventors: Raphael Beumer, Lorrach (DE); Jochen Klock, Freiburg (DE)

Correspondence Address: NIXON & VANDERHYE, PC, 901 NORTH GLEBE ROAD, 11TH FLOOR, ARLINGTON, VA, 22203, US

(21) Appl. No.: 11/990,389

(22) PCT Filed: Sep. 14, 2006
(86) PCT No.: PCT/EP2006/008962

§ 371 Date: Feb. 13, 2008

(30) Foreign Application Priority Data
Sep. 23, 2005 (EP) 05020773.7

(51) Int. Cl. A61K 8/49; A61K 8/30; A61K 31/485; A61Q 19/02; A61Q 17/04; A61P 17/02; A61K 8/64

(52) U.S. Cl. 424/59; 424/62; 514/2; 514/315; 514/282

(57) ABSTRACT

The present invention relates to a new use of opioid receptor antagonists. More particularly, the present invention relates to the use of opioid receptor antagonists for the manufacture of topical compositions for suppression of melanin formation in the human skin; to topical, particularly cosmetic, compositions for suppression of melanin formation in the human skin comprising an opioid receptor antagonist; and to a method of lightening human skin and/or treatment of pigmentation disorders which comprises topically administering an effective amount of an opioid receptor antagonist to the appropriate skin area of a person in need of such treatment.

The present invention relates to a new use of opioid receptor antagonists. More particularly, the present invention relates to the use of opioid receptor antagonists for the manufacture of topical compositions for suppression of melanin formation in the human skin; to topical, particularly cosmetic, compositions for suppression of melanin formation in the human skin comprising an opioid receptor antagonist; and to a method of lightening human skin and/or treatment of pigmentation disorders which comprises topically administering an effective amount of an opioid receptor antagonist to the appropriate skin area of a person in need of such treatment.

In accordance with the invention it has been found that antagonists of opioid receptors, particularly μ-opioid receptors, reduce the intracellular melanin production and, therefore, can be used against unwanted pigmentation of human skin and/or for the treatment of pigmentation disorders, especially as agents for skin lightening and tanning prevention in compositions for topical application, particularly for cosmetics purposes.

Such cosmetics purposes may be prevention of repigmentation, protection against sun or UV-induced skin darkening and reducing skin melanin level or enhancement of skin bleaching action; action on blemishes.

In accordance with the invention, antagonists of opioid receptors may be used also in topical, particularly pharmaceutical, compositions, for treatment and prevention of pigmentation disorders, such as

primary hyperpigmentation disorders which include those that are nevoid, congenital or acquired such as local hyperpigmented disorders which include pigmented nevi, ephelides juvenile freckles, an inherited characteristic; age spots; and café-au-lait spots) and lentigines (solar lentigines, senile lentigines, senile freckles, liver-spots); and secondary hyperpigmentation disorders which include those occurring after a separate dermatologic condition, including acne; such disorders are most commonly seen in dark-skinned individuals and are called postinflammatory hyperpigmentation.

Further hyperpigmentation disorders which include arsenical melanosis and disorders associated with Addison's disease; freckling and café-au-lait spots produced by neurofibromatosis; regional or patterned hyperpigmentation caused by melanocytic hyperactivity, such as idiopathic melasma occurring either during pregnancy or secondary to estrogen-progesterone contraception.

Other examples for disorders which, in accordance with the invention, may be treated or prevented by topical application of opioid receptor antagonists include the pigmentation following physical trauma, eczematoid eruptions are lupus erythematosus, and dermatoses such as pityriasis rosea, psoriasis, dermatitis herpetiformis, fixed drug eruptions, photodermatitis and Lichen simplex chronicus, tinea versicolor (under specific environmental conditions for a yeast type of skin fungus, present on normal skin) and Acanthosis nigricans; post-inflammatory hyperpigmentations which can result due to abrasion, burns, wounds, insect bites, dermatitis, and other similar small, fixed pigmented lesions; Berloque hyperpigmentation, which is due to phototoxicity from chemicals in the rinds of limes and other citrus fruits, and to celery; and accidental hyperpigmentation which can result from post-lesional photosensitization and scarring.

Further examples of pigmentation disorders include those caused by some drugs, including chloroquine, chlorpromazine, minocycline and amiodarone. Benzoyl peroxide, fluorouracil and tretinoin can cause hyperpigmentation; fixed drug eruptions can result from phenolphthalein in laxatives, trimethoprim-sulfamethoxazole, nonsteroidal anti-inflammatory drugs (NSAIDs) and tetracyclines.

In certain forms of leukoderma such as vitiligo where, if the injured skin cannot be repigmented, the residual zone of normal skin are depigmented to impart a homogeneous white colour to the entire skin. In all these conditions treatment in accordance with the invention may be considered.

Especially, the opioid receptor antagonists can be used in accordance with the present invention, to reduce immune suppression (e.g., after UV irradiation), exert analgesic effects (e.g., in sunburn or skin irritation), and reduce sebum production so that they may find use also in the topical treatment of acne and skin impurities.

While μ-opioid receptor antagonists are preferred for the purposes of the present invention, antagonists binding selectively to κ- or δ-opioid receptors, or binding unselectively to μ- and/or κ- and/or δ-opioid receptors may find use.

In a further aspect, the present invention relates to a method of identifying an agent which can be used reduce melanin in the human skin which comprises determining the activity of said agent to inhibit an opioid receptor, in particular a μ-opioid receptor. Thus the present invention provides a tool to identify agents which on topical application reduce the intracellular melanin production and can thus be used in topical compositions to achieve the cosmetic or therapeutical effects set forth above. Any method to determine the activity of an agent in inhibiting opioid receptor signalling can be used for the purposes of that aspect of the invention but is not limited to that. Such methods are disclosed, e.g., in J. Invest. Dermatol. 111:297-311, 1998; WO 2004/051264, WO 2004/038005, WO 2004/014310, WO 2004/005294, WO 03/101963 and WO 02/098422.

Particularly, the activity of an agent in inhibiting an opioid receptor can be further determined by measuring the efficacy of the agent to cause a substantial reduction of the total intracellular melanin production in human primary melanocytes, preferably from skin type IV. A reduction of the total intracellular melanin production of at least 20% is regarded to be indicative for a usefulness of an agent for inhibiting melanin formation and for skin lightening when applied as a topical composition.

The term “antagonists of opioid receptors” as used herein denotes any compound which inhibits the opioid receptor signalling or down-regulates the expression of the opioid receptors in human skin cells in particular melanocytes but not limited to them. The opioid receptor antagonists may be opioid analogues, e.g., (CAS number given in parenthesis where appropriate) Naloxone (465-65-6); Naloxonazine (82824-01-9); Cyprodime (118111-54-9); β-Funaltrexamine (72782-05-9); Nalbuphine (20594-83-6); RX 8008M (40994-80-7); SDZ 210-096 (109026-86-0); Clocinnamox (117332-69-1); NIH 10236 (88167-37-7); BU 165 (173321-27-2); BU 164 (173429-52-2); BU 158 (173429-53-3); BU 160 (173429-56-6); BU 161 (173429-57-7); BU 162 (173429-58-8); Buprenorphine (52485-79-7); IOXY (141392-28-1); NPC 168 (115160-07-1); Naloxazon (73674-85-8); N-Methylnaloxonium Iodide (93302-47-7); 3-Methoxynaltrexone Hydrochloride; 7-Benzylidenenaltrexone 129468-28-6); Naltrindole Isothiocyanate (126876-64-0); BNTX (153611-34-8); Naltriben (111555-58-9); Naltrexone (16590-41-3); Nalmefene (55096-26-9); β-Chlornaltrexamine (67025-94-9); Diprenorphine (14357-78-9); nor-Binaltorphimine (105618-27-7); Naltrindole (111555-53-4); or (poly)peptides, e.g., CTAP (103429-32-9); TCTOP (115981-70-9); TCTAP (115981-71-0); CTOP (103429-31-8); Tyr MIF-1 (77133-61-0); CCK-8 (25126-32-3); CG 3703 (90243-66-6); compounds disclosed in Peptide Research 1995, 8(3), 124-37, Proceedings of the National Academy of Sciences of the United States of America (1993), 90(22), 10811-15, Regulatory Peptides (1994), (Suppl. 1), S53-S54; SMS 201-995 (83150-76-9); e-PMTC as disclosed in Medicinal Chemistry Research (1994), 4(4), 245-53; CTP (103335-28-0); TIPP (146369-65-5); ICI 154129 (83420-94-4); ICI 174864 (89352-67-0); or piperidine derivatives, e.g., the compounds disclosed in J. Med. Chem. 1993, 36(20); 2833-41, EP 657428 and EP 506478; or may belong to different structures, such as Quadazocine (71276-43-2); Flumazenil (78755-81-4); BIT (85951-65-1); Dezocine (53648-55-8); Ciramadol (63269-31-8). Ginseng root extract like in Journal of Ethnopharmacology (1994), 42(1), 45-51; Rimcazole (75859-04-0); MR 2266 (56649-76-4); and WIN 44441-3 (71276-44-3).

Further opioid receptor antagonists, particularly the μ-receptor, and assays for determining their efficacy in binding to the receptors have been disclosed in e.g., WO 02/098422, U.S. Pat. No. 5,270,328, US 2001/0036951 A, WO 01/42207, WO 01/37785, WO 01/41705, WO 03/101963, WO 2004/005294, WO 2004/014310, WO 2004/038005, and WO 2004/051264.

Typical μ-opioid receptor antagonists for use according to the invention include, but are not limited to, Naloxone; Naloxonazine; piperidine derivatives such as quoted above; Cyprodime; β-Funaltrexamine, Nalbuphine, CTAP, TCTOP, TCTAP, CTOP, Quadazocine, Flumazenil, RX 8008M, SDZ 210-096, Tyr MIF-1, CCK-8, CG 3703, Clocinnamox, peptides such as disclosed in Peptide Research 1995, 8(3), 124-37, Proceedings of the National Academy of Sciences of the United States of America (1993), 90(22), 10811-15, Regulatory Peptides (1994), (Suppl. 1), S53-S54; NIH 10236, BU 165, BU 164, BU 158, BU 160, BU 161, BU 162, Buprenorphine, IOXY, SMS 201-995, e-PMTC as disclosed in Medicinal Chemistry Research (1994), 4(4), 245-53; CTP, BIT, NPC 168, Naloxazon, Dezocine and Ciramadol. Other, typical κ-, δ-receptor or non-selective (still binding to μ-receptors as well) antagonists for use herein include, but are not limited to: N-Methylnaloxonium Iodide, 3-Methoxynaltrexone Hydrochloride; 7-Benzylidenenaltrexone, Ginseng root extract as disclosed in Journal of Ethnopharmacology (1994), 42(1), 45-51; Rimcazole, Naltrindole Isothiocyanate, BNTX, TIPP, Naltriben, Naltrexone, ICI 154129, MR 2266, WIN 44441-3, Nalmefene, β-Chlornaltrexamine, ICI 174864, Diprenorphine, nor-Binaltorphimine and Naltrindole.

Further opioid receptor antagonists which have been detected during the screening as outlined in the examples and which can be used for the purposes of the present invention comprise Epigallocatechin 3,5-Digallate (37484-73-4), Irigenol Hexaacetate (103652-04-6), Irigenol ex Iris spp (4935-93-7), Berbamine Hydrochloride (5956-76-3), Quercetagetin (90-18-6), Acetylshikonin (24502-78-1), 2′,3′,4′,3,4-Pentahydroxychalcone (484-76-4), beta,beta-Dimethylacryl shikonin (24502-79-2), 2,3-Dimethoxy-5-methyl-1,4-benzoquinone (605-94-7), 2,3-Dimethoxy-5-methylhydroquinone (3066-90-8), 2,3-Dimethoxy-1,4-benzoquinone (3117-02-0), 2,3-Dimethoxyhydroquinone (52643-52-4), Delphinidin chloride (528-53-0), Aureusidin (38216-54-5), Isocembrol (25269-17-4) and Robinetin (490-31-3) without being limited thereto.

Further opioid receptor antagonists which can be used for the purposes of the present invention are disclosed in JP 63290897, U.S. Pat. No. 4,906,655, WO 9302707, CA 2064373, U.S. Pat. No. 5,270,220, U.S. Pat. No. 5,352,680, WO 9504734, WO 9513071, EP 657428, WO 9606855, WO 9640208, U.S. Pat. No. 5,641,861, WO 9733174, DE 19622866, U.S. Pat. No. 5,919,897, U.S. Pat. No. 5,948,807, WO 9945925, WO 2000008027, WO 2001037785, WO 2001041705, WO 2001042207, WO 2001046198, WO 2001068080, US 2001036951, WO 2002053533, WO 2003020277, WO 2003035622, WO 2003035645, WO 2003066050, WO 2003101963, WO 2004014310, WO 2004026305, WO 2004033458, US 2004186135, WO 2004080968, WO 2004080996, US 2004204445, WO 2004091593, WO 2004099194, US 2004254156, WO 2005003131, WO 2005030722 the contents of which are included herein by reference.

The above-identified opioid receptor antagonists can suitably be used in the form of physiologically acceptable salts, with inorganic acids, e.g. hydrochlorides, hydrobromides, sulfates, phosphates, or organic acids, e.g. methanesulfonates, p-toluenesulfonates, carbonates, formats, acetates, oxalates, lactates; or as hydrates as appropriate.

The above-identified opioid receptor antagonists or their salts may be used as racemates or as pure enantiomers, or diastereomers or mixtures thereof. Preferably, pure enantiomers are used. If one or more chiral centers are present the optical purity of the mixture is preferably ≧80% ee, more preferably ≧90% ee, most preferably ≧95% de. If two or more chiral centers are present the purity of the mixture is preferably ≧80% de, more preferably ≧90% de, most preferably ≧95% de.

In all embodiments of the invention the term ‘opioid receptor antagonists’ also encompasses any material or extract of a plant containing at least one opioid receptor antagonist of in an amount of at least 30 weight-% (i.e. from 30 to 100 weight-%), preferably in an amount of at least 50 weight-% (i.e. from 50 to 100 weight-%), more preferably in an amount of at least 70 weight-% (i.e. from 70 to 100 weight-%), most preferably in an amount of at least 90 weight-% (i.e. from 90 to 100 weight-%), based on the total weight of the plant material or extract. The terms “material of a plant” and “plant material” used in the context of the present invention mean any part of a plant.

Further, derivatives of these compounds as appropriate, such as esters, amides, nitriles, oximes, imines, hydrazones, ethers, acetals, semiacetals may also find use. The ester or ether groups may for example be derived from straight or branched alkyl groups having 1 to 26 carbon atoms or from substituted or unsubstituted straight or branched aliphatic, araliphatic or aromatic carboxylic acids having 1 to 26 carbon atoms. Examples of etherified hydroxy groups further include glycoside groups. Examples of esterified hydroxy group further include glucuronide or sulfate groups.

Of particular interest for the purposes of the present invention are the opioid receptor antagonists naloxone, naloxonazine, isocembrol, 2′,3′,4′,3,4-pentahydroxychalcone, aureusidin or 2,3-dimethoxy-5-methylhydroquinone, in particular naloxone, isocembrol, 2′, 3′,4′,3,4-pentahydroxychalcone, aureusidin and 2,3-dimethoxy-5-methylhydroquinone.

In accordance with the present invention, the antagonists of opioid receptors with the definitions and preferences as given above can be used in topical compositions, in particular topical cosmetic or pharmaceutical compositions, for reducing melanin in the human skin, for lightening and tanning prevention of the human skin and/or for the treatment and prevention of pigmentation disorders. Thus, the invention also relates to a topical composition for reducing melanin in the human skin, for lightening and tanning prevention of the human skin and/or for the treatment and prevention of pigmentation disorders comprising at least one antagonist an opioid receptor antagonist and carriers and/or excipients or diluents conventionally used in topical compositions. If nothing else is stated, the excipients, additives, diluents, etc. mentioned in the following are suitable for both pharmaceutical and cosmetic compositions. The necessary amounts of the cosmetic and dermatological adjuvants and additives can, based on the desired product, easily be determined by the skilled person.

In another embodiment the invention relates to a topical composition comprising an opioid receptor antagonists with the definitions and preferences as given above and at least one additional skin lightening and/or UV-screening agent, and a conventional carrier.

In all embodiments of the invention the compositions according to the invention comprise the opioid receptor antagonist in an amount of at least 0.00001 wt.-%. Preferably from about 0.00001 wt.-% to about 20 wt.-%, more preferably from about from about 0.0001 wt.-% to about 10 wt.-%, most preferably from about from about 0.005 wt.-% to about 5 wt.-%.

Furthermore the invention relates to a method of lightening human skin which comprises topically administering an effective amount of an opioid receptor antagonist with the definitions and preferences as given above, in particular an antagonist of μ-opioid receptors, to the appropriate skin area of a person in need of such treatment. Additionally, the invention relates to a method of treating or preventing pigmentation disorders which comprises topically administering an effective amount of an opioid receptor antagonist to the appropriate skin area of a person in need of such treatment.

The term ‘an effective amount’ refers to an amount necessary to obtain a physiological effect. The physiological effect may be achieved by one application dose or by repeated applications. The dosage administered may, of course, vary depending upon known factors, such as the physiological characteristics of the particular composition comprising the fatty acid or a salt, ester or amide with the definitions and preferences as given above, optionally in combination with a retinoid and its mode and route of administration; the age, health and weight of the recipient; the nature and extent of the symptoms; the kind of concurrent treatment; the frequency of treatment; and the effect desired and can be adjusted by a person skilled in the art.

Preferably, the invention relates to a method according to the invention wherein from about 0.2 μg to about 200 μg of opioid receptor antagonist are applied per square centimetre of skin per day.

Preferred topical compositions are topical cosmetic or pharmaceutical compositions intended for the application onto human skin.

The term “cosmetic preparation” or “cosmetic composition” as used in the present invention e.g. refers to cosmetic compositions as defined under the heading “Kosmetika” in Römpp Lexikon Chemie, 10th edition 1997, Georg Thieme Verlag Stuttgart, New York. Regarding the kind of the topical cosmetic and pharmaceutical composition and the preparation of the topical cosmetic and pharmaceutical preparations as well as for further suitable additives, it can be referred to the pertinent literature, e.g. to Novak G. A., Die kosmetischen Präparate-Band 2, Die kosmetischen Präparate—Rezeptur, Rohstoffe, wissenschaftliche Grundlagen (Verlag für Chem. Industrie H. Ziolkowski K G, Augsburg).

Preferably, the topical cosmetic or pharmaceutical compositions of the present invention are in the form of a suspension or dispersion in solvents or fatty substances, or alternatively in the form of an emulsion or micro emulsion (in particular of O/W or W/O type, O/W/O or W/O/W-type), PET-emulsions, multiple emulsions, bickering emulsions, hydrogels, alcoholic gels, lipogels, one or multiphase solutions or a vesicular dispersion and other usual compositions, which can also be applied by pens, as masks or as sprays. The emulsions can also contain anionic, nonionic, cationic or amphoteric surfactant(s).

Preferred topical cosmetic or pharmaceutical compositions according to the invention are skin care preparations, decorative preparations, light protection preparations and functional preparations.

Examples of skin care preparations are, in particular, body oils, body lotions, body gels, treatment creams, skin protection ointments, shaving preparations, such as shaving foams or gels, skin powders such as baby powder, moisturizing gels, moisturizing sprays, revitalizing body sprays, cellulite gels, anti acne preparations and peeling preparations.

Examples of decorative preparations are in particular lipstick, eye shadow, mascaras, dry and moist make-up, rouge, powders, and suntan lotions.

Examples of functional preparations are topical cosmetic or pharmaceutical compositions containing active ingredients such as hormone preparations, vitamin preparations, vegetable extract preparations, anti-ageing preparations, and antimicrobial (antibacterial or antifungal) preparations without being limited thereto.

Cosmetic compositions in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as a aerosol mousse, a foam or a spray foams, sprays, sticks, a gel, a plaster, a powder, a cleanser, a soap or aerosols or wipes. Preferred topical compositions comprise a cream, a gel, an ointment, a lotion a tincture, a spray, a mousse, a cleansing composition or foam.

The topical cosmetic or pharmaceutical compositions of the invention can also contain usual cosmetic or pharmaceutical adjuvants and additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, e.g. those suited for providing a photoprotective effect by physically blocking out ultraviolet radiation, or any other ingredients usually formulated into topical cosmetic or pharmaceutical compositions. The necessary amounts of the cosmetic and dermatological adjuvants and additives can, based on the desired product, easily be chosen by a skilled artisan in this field and will be illustrated in the examples, without being limited hereto. The usual cosmetic adjuvants and additives such as emulsifiers, thickeners, surface active ingredients and film formers can show synergistic which can be determined by the expert in the field with normal trials, or with the usual considerations regarding the formulation of topical cosmetic or pharmaceutical composition.

An additional amount of antioxidants/preservatives is generally preferred. Based on the invention all known antioxidants usually formulated into topical cosmetic or pharmaceutical compositions can be used. Especially preferred are antioxidants chosen from the group consisting of amino acids (e.g. glycine, histidine, tyrosine, tryptophan) and their derivatives, imidazole (e.g. urocanic acid) and derivatives, peptides such as D,L-carnosine, D-carnosine, L-carnosine and derivatives (e.g. anserine), carotenoids, carotenes (e.g. α-carotene, β-carotene, lycopene) and derivatives, chlorogenic acid and derivatives, lipoic acid and derivatives (e.g. dihydrolipoic acid), aurothioglucose, propylthiouracil and other thiols (e.g. thioredoxin, glutathione, cysteine, cystine, cystamine and its glycosyl-, N-acetyl-, methyl-, ethyl-, propyl-, amyl-, butyl- and lauryl-, palmitoyl-; oleyl-, y-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinesulfoximine, buthioninsulfone, penta-, hexa-, heptathioninsulfoximine) in very low compatible doses (e.g. pmol to μmol/kg), additionally (metal)-chelators (such as α-hydroxyfatty acids, palmic-, phytinic acid, lactoferrin), β-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyltetraisopalmitate, Mg-ascorbylphosphate, Na-ascorbylphosphate, Na-ascorbylacetate), tocopherol and derivatives (such as vitamin-E-acetate), mixtures of nat. vitamin E, vitamin A and derivatives (vitamin-A-palmitate and -acetate) as well as coniferylbenzoate, rutinic acid and derivatives, α-glycosylrutin, ferulic acid, furfurylideneglucitol, carnosine, butylhydroxytoluene, butylhydroxyanisole, trihydroxy-butyrophenone, urea and its derivatives, mannose and derivatives, zinc and derivatives (e.g. ZnO, ZnSO₄), selenium and derivatives (e.g. selenomethionin), stilbenes and derivatives (such as stilbenoxide, trans-stilbenoxide) and suitable derivatives (salts, esters, ethers, sugars, nucleotides, nucleosides, peptides and lipids) of the named active ingredients. One or more preservatives/antioxidants may be present in an amount about 0.01 wt. % to about 10 wt. % of the total weight of the topical cosmetic or pharmaceutical topical composition of the present invention. Preferably, one or more preservatives/antioxidants are present in an amount about 0.1 wt. % to about 1 wt. %.

Typically topical cosmetic or pharmaceutical compositions also contain surface active ingredients like emulsifiers, solubilizers and the like. An emulsifier enables two or more immiscible components to be combined homogeneously. Moreover, the emulsifier acts to stabilize the composition. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polyglyceryl-4-oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glyceride phosphates and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. The preferred emulsifiers are cetyl phosphate (Amphisol® A), diethanolamine cetyl phosphate (Amphisol®), potassium cetyl phosphate (Amphisol® K), PVP Eicosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount about 0.01 wt. % to about 20 wt. % of the total weight of the topical cosmetic or pharmaceutical topical composition of the present invention. Preferably, about 0.1 wt. % to about 10 wt. % of emulsifiers is used.

The lipid phase of the topical cosmetic or pharmaceutical compositions can advantageously be chosen from: mineral oils and mineral waxes; oils such as triglycerides of caprinic acid or caprylic acid and castor oil; oils or waxes and other natural or synthetic oils, in a preferred embodiment esters of fatty acids with alcohols e.g. isopropanol, propylene glycol, glycerin or esters of fatty alcohols with carboxylic acids or fatty acids; alkylbenzoates; and/or silicone oils such as dimethylpolysiloxane, diethylpolysiloxane, diphenylpolysiloxane, cyclomethicones and mixtures thereof.

Exemplary fatty substances which can be incorporated in the oil phase of the emulsion, microemulsion, oleo gel, hydrodispersion or lipodispersion of the topical cosmetic or pharmaceutical composition of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaureate, n-decyloleate, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the topical cosmetic or pharmaceutical compositions of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicones (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in topical cosmetic or pharmaceutical compositions of the present invention are isoeikosane; neopentylglycoldiheptanoate; propyleneglycoldicaprylate/dicaprate; caprylic/capric/diglycerylsuccinate; butyleneglycol caprylat/caprat; C₁₂₋₁₃-alkyllactate; di-C₁₂₋₁₃ alkyltartrate; triisostearin; dipentaerythrityl hexacaprylat/hexacaprate; propylene-glycolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the topical cosmetic or pharmaceutical compositions of the present invention can also contain natural vegetable or animal waxes such as bee wax, china wax, bumblebee wax and other waxes of insects as well as shea butter and cocoa butter.

A moisturizing agent may be incorporated into a topical cosmetic or pharmaceutical composition of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 20 wt. % of the total weight of the topical cosmetic or pharmaceutical composition. The preferred amount of emollient is about 2 wt. % to about 15 wt. %, and most preferably about 4 wt. % to about 10 wt. %.

Moisturizers that bind water, thereby retaining it on the skin surface are called humectants. Suitable humectants can be incorporated into a topical cosmetic or pharmaceutical composition of the present invention such as glycerin, polypropylene glycol, polyethylene glycol, lactic acid, pyrrolidone carboxylic acid, urea, phospholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel®1000 (CAS-Nr. 178463-23-5) by SOLABIA S. One or more humectants are optionally present at about 0.5 wt. % to about 8 wt. % in a topical cosmetic or pharmaceutical composition of the present invention, preferably about 1 wt. % to about 5 wt. %.

The aqueous phase of the preferred topical cosmetic or pharmaceutical compositions of the present invention can contain the usual cosmetic or pharmaceutical additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, foam stabilizers; electrolytes and especially one or more thickeners. However, preferably the topical cosmetic or pharmaceutical compositions of the present invention are free of ethanol, more preferably they are free of alcohols, and most preferably they are free of organic solvents, since such compounds can cause skin irritation.

Thickeners that may be used in topical cosmetic or pharmaceutical compositions of the present invention to assist in making the consistency of a product suitable include carbomer, siliciumdioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as Carbopole® of type 980, 981, 1382, 2984, 5984 alone or mixtures thereof.

Suitable neutralizing agents which may be included in the topical cosmetic or pharmaceutical composition of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the topical cosmetic or pharmaceutical composition of the present invention, preferably, 1 wt. % to about 5 wt. %.

The addition of electrolytes into the topical cosmetic or pharmaceutical composition of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the topical cosmetic or pharmaceutical composition of the present invention.

The topical cosmetic or pharmaceutical compositions of the present invention is preferably applied at least once per day, e.g. twice or triple times a day.

The amount of the topical cosmetic or pharmaceutical composition, which is to be applied to the skin, depends on the concentration of the opioid antagonist and optionally other active ingredients in the compositions and the desired cosmetic or pharmaceutical effect. For example, application can be such that a crème is applied to the skin. A crème is usually applied in an amount of 2 mg crème/cm² skin. The amount of the topical cosmetic or pharmaceutical composition which is applied to the skin is, however, not critical, and if with a certain amount of applied topical cosmetic or pharmaceutical composition the desired effect cannot be achieved, a higher concentration of the opioid antagonist can be used e.g. by applying more of the topical cosmetic or pharmaceutical composition or by applying topical cosmetic or pharmaceutical compositions which contain more opioid antagonist.

The composition according to the invention can also contain one or more additional pharmaceutically or cosmetically active ingredient, in particular for skin lightening, tanning prevention, treatment of hyperpigmentation, preventing or reducing acne, wrinkles, lines, atrophy, inflammation, as well as topical anesthetics, antimicrobial agents, and antifungal agents, chelators and sequestrants; anti-cellulites agents and sunscreening additives. Examples of such ingredients are peptides (e.g., Matrixyl™ [pentapeptide derivative]), oligopeptides, wax-based synthetic peptides (e.g., octyl palmitate and tribehenin and sorbitan isostearate and palmitoyl-oligopeptide), glycerol, urea, guanidine (e.g. amino guanidine); vitamins and derivatives thereof such as vitamin C (ascorbic acid), vitamin A (e.g., retinoid derivatives such as retinyl palmitate or retinyl propionate), vitamin E (e.g., tocopherol acetate), vitamin B₃ (e.g. niacinamide) and vitamin B₅ (e.g. panthenol), vitamin B₆ and vitamin B₁₂, biotin, folic acid; anti-acne actives or medicaments (e.g. resorcinol, salicylic acid, and the like); antioxidants (e.g. phytosterols, lipoic acid); flavonoids (e.g. isoflavones, phytoestrogens); skin soothing and healing agents such as aloe vera extract, allantoin and the like; agents suitable for aesthetic purposes such as essential oils, fragrances, skin sensates, opacifiers, aromatic compounds (e.g., clove oil, menthol, camphor, eucalyptus oil, and eugenol), desquamatory actives, hydroxy acids such as AHA acids, radical scavengers, farnesol, antifungal actives in particular bisabolol, alkyldiols such as 1,2-pentanediol, hexanediol or 1,2-octanediol, phytol, polyols such as phytanetriol, ceramides and pseudoceramides, amino acids, protein hydrolysates, polyunsaturated fatty acids, plant extracts like kinetin, DNA or RNA and their fragmentation products, carbohydrates, conjugated fatty acids, carnitin, carnosine, biochinonen, phytofluen, phytoen, and their corresponding derivatives.

According to a further embodiment of the invention, the opioid receptor antagonists can be used in combination with one or more agents selected from skin lightening agents and UV screening agents. Accordingly, the present invention is also concerned with novel topical cosmetic or pharmaceutical compositions comprising an opioid receptor antagonist and at least one additional skin lightening agent and/or a UV screening agent. The use of combinations of skin lightening agents may be advantageous in that they may provide skin lightening benefit through different mechanisms. Examples of additional, other skin lightening agents, which may be present in the topical cosmetic or pharmaceutical compositions of the present invention are especially those disclosed in WO 2004/062635, WO 2004/037213, and DE 102 38 449.

Preferably, the additional skin lightening agent comprises a water soluble skin lightening agent selected from ascorbic acid compounds, vitamin B₃ compounds, azelaic acid, gallic acid and its derivatives, hydroquinone, kojic acid, arbutin, mulberry extract, and mixtures thereof. In one preferred embodiment, a combination of ascorbic acid compounds and vitamin B₃ compounds are used. Skin lightening agents herein include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. The type and amount of skin lightening agents are selected so that the inclusion of a specific agent does not affect the stability of the composition. For example, while water-soluble agents are preferable from a composition stability point of view, water-insoluble agents may also be included to the extent it can be dispersed with the carboxylic acid/carboxylate copolymer and or optional lower alkyl alcohol carrier, and thus does not affect the stability of the present composition. The term “water soluble” with regard to skin lightening agents herein, relate to compounds that are completely dissolved to make a transparent solution when dissolved in ample amount of water at ambient temperature. Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein includes, for example, esters of ascorbic acid, and ester salts of ascorbic acid. Particularly preferred ascorbic acid compounds include 2-O-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include: magnesium ascorbyl phosphate, 2-O-D-glucopyranosyl-L-ascorbic acid, and sodium L-ascorbyl phosphate.

Vitamin B₃ compounds useful herein include, for example, niacinamide and nicotinyl alcohols, derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form.

Vitamin A acetate or palmitate which may be present in the skin care products in an amount from about 0.01 wt.-% to about 1.00 wt.-%.

The ascorbic acid derivative for use in accordance with the present invention may by any non-toxic, non skin-irritating water-soluble or oil-soluble ascorbic acid derivative. Examples of such oil soluble derivatives are ascorbyl palmitate, ascorbyl tetraisopalmitate, ascorbyl linoleate, ascorbyl octanoate. Preferred are water soluble ascorbyl derivatives such as sodium ascorbyl phosphate, magnesium ascorbyl phosphate and ascorbyl glycoside. The amount of ascorbic acid derivative in the skin care product for use in accordance with the present invention is suitably in the range from about 0.1 wt.-% to about 5 wt.-%.

A vitamin E derivative for use in the present invention is tocopheryl acetate. Tocopheryl acetate may be present in the skin care products in an amount from about 0.05 wt.-% to about 5 wt.-%. Another vitamin E derivative of interest is tocopheryl linoleate. Tocopheryl linoleate may be present in the skin care composition in an amount from about 0.05 wt.-% to about 5 wt.-%.

Examples of vitamins from the B complex for use in the present invention are vitamin B₃, B₆ and biotin. Vitamin B₃ may be present in the skin care products in an amount from about 0.01 wt.-% to about 1.00 wt.-%. Vitamin B₆ may be present in the skin care products in an amount from about 0.01 wt-% to about 1.00 wt.-%. Biotin may be present in the skin care products in an amount from about 0.001 wt.-% to about 0.5 wt.-%.

Panthenol may be present in the skin care products in an amount from about 0.05 wt.-% to about 5.00 wt.-%. Phytantriol may be present in the skin care products in an amount from about 0.01 wt.-% to about 2.5 wt.-%. Bisabolol may be present in the skin care products in an amount from about 0.05 wt.-% to about 5.00 wt.-%.

Further skin lightening agents which may be used in the present invention in combination with an opioid receptor antagonist are

### Bis-pantoyl-cystamine;

Kojic acid or derivatives thereof, which may be present in the skin care products of the present invention in an amount from about 0.05 wt.-% to about 5 wt.-%;
Arbutin or derivatives thereof which may be present in the skin care products of the present invention in an amount from about 0.05 wt.-% to about 5 wt.-%;
Hydroquinone or derivatives thereof which may be present in the skin care products of the present invention in an amount from about 0.05 wt.-% to about 2 wt.-%;
Phyllanthus Emblica fruit extract (trade name: Emblica™), which may be present in the skin care compositions of the present invention in an amount from 0.05 wt.-% to about 3 wt.-%;
Leucocyte extract, which may be present in the skin care compositions of the present invention in an amount from 0.05 wt.-% to about 3 wt.-%;
Bearberry extract, which may be present in the skin care compositions of the present invention in an amount from 0.05 wt.-% to about 3 wt.-%;
Licorice extract, which may be present in the skin care compositions of the present invention in an amount from 0.05 wt.-% to about 3 wt.-%; and
Mulberry extract, which may be present in the skin care compositions of the present invention in an amount from 0.05 wt.-% to about 3 wt.-%.

A safe and effective amount of a desquamation active may be added to the compositions of the present invention, more preferably from about 0.1% to about 10%, even more preferably from about 0.2% to about 5%, by weight of the composition. Desquamation actives enhance the skin appearance benefits of the present invention. One desquamation system that is suitable for use herein contains sulfhydryl compounds and zwitterionic surfactants and is described in U.S. Pat. No. 5,681,852. Another desquamation system that is suitable for use herein contains salicylic acid and zwitterionic surfactants and is described in U.S. Pat. No. 5,652,228. Zwitterionic surfactants such as described in these applications are also useful as desquamatory agents herein, with cetyl betaine being particularly preferred.

According to the invention for preparing the compositions the active ingredients can be used as such or in an encapsulated form, for example in a liposomal form. Liposomes are preferably formed with lecithins with or without addition of sterols or phytosterols. The encapsulation of the active ingredients can be alone or together with other active ingredients. Other embodiments include solid or semisolid capsules aiming to protect the retinoid from degradation or for controlled delivery. Suitable encapsulation technologies are for example described in WO 0180823, WO 9903450, WO 9317784 or in Fragrance Journal (2001), 29(2), 83-90.

Additionally the cosmetic and pharmaceutical topical composition of the present invention may contain UV-screening agents. The additional UV-screening agents are advantageously selected from IR, UV-A, UV-B, UV-C and/or broadband filters. Examples of UV-B or broad spectrum screening agents, i.e. substances having absorption maximums between about 290 nm and 340 nm may be organic or inorganic compounds. Organic UV-B or broadband screening agents are e.g. acrylates such as 2-ethylhexyl 2-cyano-3,3-diphenylacrylate (octocrylene, PARSOL® 340), ethyl 2-cyano-3,3-diphenylacrylate and the like; camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, therephthalidene dicamphor sulfonic acid and the like; Cinnamate derivatives such as ethylhexyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes; p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate; benzophenones such as benzophenone-3, benzophenone-4, 2,2′,4,4′-tetrahydroxy-benzophenone, 2,2′-dihydroxy-4,4′-dimethoxybenzophenone and the like; esters of benzalmalonic acid such as di-(2-ethylhexyl) 4-methoxybenzalmalonate; esters of 2-(4-ethoxy-anilinomethylene)propandioic acid such as 2-(4-ethoxy anilinomethylene) propandioic acid diethyl ester as described in the European Patent Publication EP 0895 776; organosiloxane compounds containing benzmalonate groups as described in the European Patent Publications EP 0358584 B1, EP 0538431 B1 and EP 0709080 A1 such as PARSOL® SLX; drometrizole trisiloxane (Mexoryl XL); imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like; salicylate derivatives such as isopropylbenzyl salicylate, benzyl salicylate, butyl salicylate, ethylhexyl salicylate (PARSOL® EHS, Neo Heliopan OS), isooctyl salicylate or homomethyl salicylate (homosalate, PARSOL® HMS, Neo Heliopan HMS) and the like; triazine derivatives such as ethylhexyl triazone (Uvinul T-150), diethylhexyl butamido triazone (Uvasorb HEB) and the like. Encapsulated UV-filters such as encapsulated ethylhexyl methoxycinnamate (Eusolex UV-pearls) or microcapsules loaded with UV-filters as e.g. disclosed in EP 1471995 and the like;

Examples of broad spectrum or UV A screening agents i.e. substances having absorption maximums between about 320 nm and 400 nm may be organic or inorganic compounds. Organic broad spectrum or UV A screening agents include e.g. dibenzoylmethane derivatives such as 4-tert.-butyl-4′-methoxydibenzoyl-methane (PARSOL® 1789), dimethoxydibenzoylmethane, isopropyldibenzoylmethane and the like; benzotriazole derivatives such as 2,2′-methylene-bis-(6-(2H-benzotriazole-2-yl)-4-(1,1,3,3,-tetramethylbutyl)-phenol (Tinosorb M) and the like; bis-ethylhexyloxyphenol methoxyphenyl triazine (Tinosorb S) and the like; phenylene-1,4-bis-benzimidazolsulfonic acids or salts such as 2,2-(1,4-phenylene)bis-(1H-benzimidazol-4,6-disulfonic acid) (Neoheliopan AP); amino substituted hydroxybenzophenones such as 2-(4-Diethylamino-2-hydroxy-benzoyl)-benzoic acid hexylester (Uvinul A plus) as described in the European Patent Publication EP 1046391; Ionic UV-A filters as described in the International Patent Publication WO2005080341 A1; As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g. 3,3-Diphenylacrylate derivatives as described in the European Patent Publications EP 0 514 491 B1 and EP 0 780 119 A1; Benzylidene camphor derivatives as described in the U.S. Pat. No. 5,605,680; Organosiloxanes containing benzmalonate groups as described in the European Patent Publications EP 0358584 B1, EP 0538431 B1 and EP 0709080 A1.

A good overview of UV-A- and UV-B screening agents which can be added to the compositions of the present invention can also be found in DE-A 103 27 432. All UV-filter compounds disclosed in this document are also useful as components for the compositions of the present invention and are included herein by reference. A safe and effective amount of the UV-screening agent is used, typically from about 1 wt.-% to about 20 wt.-%, more typically from about 2 wt.-% to about 10 wt.-%.

Other suitable UV-screening agents which may be incorporated into the topical cosmetic or pharmaceutical compositions of the present invention are inorganic pigments such as microparticulated metal oxides (e.g. PARSOL® TX). Examples of such compounds include e.g. titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. The metal oxide particles may also be coated by metal oxides such as e.g. aluminum or zirconium oxides or by organic coatings such as e.g. polyols, methicone, aluminum stearate, alkyl silane. Such coatings are well known in the art. When used herein, the inorganic sunscreens are present in the amount of from about 0.1 wt.-% to about 20 wt.-%, preferably from about 0.5 wt.-% to about 10 wt.-%, more preferably from about 1 wt.-% to about 5 wt.-%.

The usefulness of skin lightening agent for tanning prevention, to lightening natural skin color or to brighten hyperpigmented skin areas can be determined by methods known in the art, see, e.g., Greatens, A., et al., Effective inhibition of melanosome transfer to keratinocytes by lectins and niacinamide is reversible. Experimental Dermatology, 2005. 14(7): p. 498-508; Hakozaki, T., et al., The effect of niacinamide on reducing cutaneous pigmentation and suppression of melanosome transfer. British journal of dermatology FIELD Publication Date: 2002 July, 2002. 147(1): p. 20-31; Griffiths, C. E., et al., Topical tretinoin (retinoic acid) improves melasma. A vehicle-controlled, clinical trial. British journal of dermatology FIELD Publication Date: 1993 October, 1993. 129(4): p. 415-21; and Hayakawa, R., et al., Biochemical and clinical study of calcium pantetheine-S-sulfonate. Acta vitaminologica et enzymologica FIELD Publication Date: 1985, 1985. 7(1-2): p. 109-14.

Following the clinical study design as especially mentioned at Greatens et al, supra, and Hakozaki et al., supra, an O/W skin lightening cream such as mentioned in Example 5 including the ingredient to be tested at different concentrations ranging from 0.1% to 10% or even more preferable from 1% to 5% is done. Briefly, a human clinical study is performed in a double-blinded, randomized, vehicle-controlled, split-face design with at least 30 people per group. Application is done in a dosage-controlled manner twice daily over at least 4 weeks, more preferable over 8 weeks and especially over 12 weeks. Quantification of lightening effects can be performed with several methods such as self-assessment Hakozaki et al., supra, visual assessment Greatens et al., supra, assessment by image analysis or assessment by a chromameter (Hakozaki et al., supra). Briefly, to identify a valid skin lightening agent a self-assessment should result that more than 50% of a treatment group realized at least a slightly brightening. For the visual assessment a reviewers' evaluation of at least 8 people is done with a grading from 1 to 4 and should result in a significant difference of the means with a p-value<0.05. An assessment by image analysis is done by using CCD camera system and quantifying basal skin color (L*, a*, b*) and area of hyperpigmentation (mm²) by computer analysis of the video images. Basal skin color can be quantified either as delta L*a*b* or as ITA° depending on L* and b* for brown pigmentation only. Data are only valid with a p-value<0.05. Assessment by a chromameter is done in the same way as described for image analysis using L*a*b*- and/or ITA°-values for quantification.

The following Examples are illustrative but not limitative of the invention.

### EXAMPLE 1

The usefulness of opioid receptor antagonists in reducing melanin production in the skin and, thus, to act as a depigmentation, i.e., skin lightening agent, can be shown by in vitro measuring the total intracellular melanin production in human primary melanocytes from skin type IV in which intrinsic melanin production is high.

### Culture of Human Melanocytes

Normal human melanocytes were cultured from freshly excised foreskin of skin type IV and treated with Dispase (ready-to-use, Roche) overnight at 4° C. Melanocytes dissociated from the epidermis were plated in culture flasks at high density using Melanocyte Growth Medium M2 (Promocell) supplemented with the corresponding growth factors (Promocell). These primary cells were cultivated under 100% humidity and 5% CO₂ at 37° C. At the second passage, melanocytes were transferred to 6 well plates and allowed to reach 80% surface confluence.

### Depigmentation of Human Primary Melanocytes

Cells were grown for 4 days in Melanocyte Growth Medium supplemented with Naloxone (Alltech) in known non-toxic concentrations (5×10⁻⁶ and 1×10⁻⁴ M). Medium was renewed on day two of the experiment. Cells were harvested, counted and lysed in 1N NaOH using vigorous vortexing and a 30 minute incubation in an ultrasound bath. Melanin content was determined by measuring the absorption of cell lysates at 475 nm. Values were calibrated against a standard curve based on synthetic melanin (Sigma). The amount of melanin per 1 million cells is reported. Each treatment was run in duplicate.

Naloxone dramatically reduces intracellular melanin content compared to the control as can be seen from Table 1. Melanin content in untreated cells is on average 7.39 μg per million cells, compared to 1.58 and 1.05 for those treated with 5×10⁻⁶ M and 1×10⁻⁴ M Naloxone, respectively.

TABLE 1

| | | Reduction of |
|---|---|---|
| | Melanin (μg/10⁶ cells) | mean melanin content |
| Control | 7.39 | — |
| Naloxone 5 × 10⁻⁶ M | 1.58 | 78% |
| Naloxone 1 × 10⁻⁴ M | 1.05 | 86% |

As can be seen from the data, in the higher concentration, Naloxone reduced the mean melanin content by 86% und is thus suitable as depigmentation, i.e., skin lightening agent. The following example exemplifies typical screening assays useful for the identification of opioid receptor antagonists.

### EXAMPLE 2

### μ-Opioid Receptor Binding Assay

250 μg/ml membranes from CHO cells overexpressing the μ-opioid receptor (Perkin Elmer) were incubated with gentle shaking for 2 h with 50 nM [³H] DAMGO (Amersham) in 50 mM Tris HCl pH 7.4 (Sigma), 10 mM MgCl₂ (Sigma) and 0.2% BSA (Sigma) in the presence of the negative control DMSO (Fluka) or the test compounds. The incubation mixture was transferred to filter plates (Perkin Elmer), the liquid aspirated through the application of a vacuum from the bottom and the plate washed twice with PBS (Invitrogen). The plate was allowed to dry, 35 μl of scintillation cocktail (Ultima Gold, Perkin Elmer) added, sealed and placed on a TopCount (Packard) for scintillation counting.

### μ-Opioid Receptor GTP-Recruitment Assay

10 μg/ml membranes from CHO cells overexpressing the β-opioid receptor (Perkin Elmer) were incubated with gentle shaking for 1.5 h with 100 nM DAMGO (Sigma) in 20 mM HEPES pH 7.4 (Sigma), 10 mM MgCl₂ (Sigma), 100 mM NaCl₂ (Fluka), 1 μM GDP (Sigma) and 10 nM GTP-Eu (Perkin Elmer) in the presence of the negative control DMSO (Fluka) or the test compounds. The incubation mixture was transferred to filter plates (Perkin Elmer), the liquid aspirated through the application of a vacuum from the bottom and the plate washed twice with PBS (Invitrogen) and allowed to dry. Plates were read in an Analyst reader (LJL Biosystems).

Using the methods outlined above the following p-opioid receptor antagonists have been identified:

Epigallocatechin 3,5-Digallate (37484-73-4), Irigenol Hexaacetate (103652-04-6), Irigenol ex Iris spp (4935-93-7), Berbamine Hydrochloride (5956-76-3), Quercetagetin (90-18-6), Acetylshikonin (24502-78-1), 2′,3′,4′,3,4-Pentahydroxychalcone (484-76-4), beta,beta-Dimethylacryl shikonin (24502-79-2), 2,3-Dimethoxy-5-methyl-1,4-benzoquinone (605-94-7), 2,3-Dimethoxy-5-methylhydroquinone (3066-90-8), 2,3-Dimethoxy-1,4-benzoquinone (3117-02-0), 2,3-Dimethoxyhydroquinone (52643-52-4), Delphinidin chloride (528-53-0), Aureusidin (38216-54-5), Isocembrol (25269-17-4), Robinetin (490-31-3).

The following examples are concerned with topical compositions which may be prepared by procedures known per se in the art.

### EXAMPLE 3

Skin Lightening Cream (O/W)

| Ingredients | % (w/w) |
|---|---|
| Estol 3650 (Glyceryl Myristate) | 5.00 |
| Lanette 16 (Cetyl Alcohol) | 2.00 |
| Amphisol A (Cetyl Phosphate) | 2.00 |
| Tegosoft M (Isopropyl Myristate) | 10.00 |
| Vitamin E Acetate (Tocopheryl Acetate) | 0.50 |
| Almond Oil | 2.00 |
| BHT | 0.05 |
| Phenonip (Phenoxyethanol & Methylparaben & | 0.60 |
| Ethylparaben & Propylparaben & Butylparaben | |
| & Isopropylparaben) | |
| Tris (Tromethamine) | 0.90 |
| EDETA BD (Disodium EDTA) | 0.10 |
| Propylene Glycol | 5.00 |
| Naloxone | 2.00 |
| Sepigel 305 (Polyacrylamide & C13-14 Isoparaffin | 2.00 |
| & Laureth-7) | |
| Triethanolamine | q.s. |
| Water deionized | ad 100 |

### EXAMPLE 4

Skin Lightening Cream (O/W)

| Ingredients | % (w/w) |
|---|---|
| Estol 3650 (Glyceryl Myristate) | 5.00 |
| Lanette 16 (Cetyl Alcohol) | 2.00 |
| Brij 72 (Steareth-2) | 2.00 |
| Brij 721 (Steareth-21) | 2.00 |
| Tegosoft M (Isopropyl Myristate) | 10.00 |
| Bisabolol | 0.20 |
| Vitamine E Acetate (Tocopheryl Acetate) | 1.00 |
| Almond Oil | 2.00 |
| BHT | 0.05 |
| Phenonip (Phenoxyethanol & Methylparaben & | 0.60 |
| Ethylparaben & Propylparaben & | |
| Butylparaben & Isopropylparaben) | |
| EDETA BD (Disodium EDTA) | 0.10 |
| Propylene Glycol | 10.00 |
| Sodium Ascorbyl Phosphate | 1.00 |
| Arbutin | 1.00 |
| D-Panthenol | 0.50 |
| Niacinamide | 0.20 |
| Salicylic acid | 0.50 |
| Naloxone | 1.00 |
| Sepigel 305 (Polyacrylamide & C13-14 | 2.00 |
| Isoparaffin & Laureth-7) | |
| Triethanolamine | q.s. |
| Water deionized | ad 100 |

### EXAMPLE 5

Skin lightening cream with UV protection (indicative SPF: 8, O/W)

| Ingredients | % (w/w) |
|---|---|
| Estol 3650 (Glyceryl Myristate) | 5.00 |
| Lanette 16 (Cetyl Alcohol) | 2.00 |
| Brij 72 (steareth-2) | 2.00 |
| Brij 721 (steareth-21) | 2.00 |
| Tegosoft M (Isopropyl Myristate) | 10.00 |
| BHT | 0.05 |
| Ascorbyl Palmitate | 0.50 |
| Parsol 1789 (Butyl Methoxydibenzoylmethane) | 1.00 |
| Parsol MCX (Ethyl Hexylmethoxycinnamate | 2.00 |
| Eusolex OS (Octyl Salicylate) | 2.00 |
| Phenonip (Phenoxyethanol & Methylparaben | 0.60 |
| & Ethylparaben & Propylparaben & | |
| Butylparaben & Isopropylparaben) | |
| EDETA BD (Disodium EDETA) | 0.10 |
| Naloxone | 2.00 |
| Propyleneglycol | 8.00 |
| Sepigel | 2.00 |
| Triethanolamine | q.s. |
| Water deionized | ad 100 |

### EXAMPLE 6

Skin Lightening Cream (W/O)

| Ingredients | % (w/w) |
|---|---|
| Cremophor WO7 (PEG-7 Hydrogenated Castor Oil) | 6.00 |
| Elfacos ST 9 (PEG-45/Dodecyl Glycol Copolymer) | 2.00 |
| Myritol 318 (Caprylic/Capric Triglyceride) | 5.00 |
| Lunacera M (Micro wax) | 2.00 |
| Paraffin Oil | 10.00 |
| Resveratrol | 0.50 |
| Phytantriol | 0.10 |
| Vitamine E Acetate (Tocopheryl Acetate) | 1.00 |
| Jojoba Oil | 5.00 |
| BHT | 0.05 |
| Phenonip (Phenoxyethanol & Methylparaben & | 0.60 |
| Ethylparaben & Propylparaben & | |
| Butylparaben & Isopropylparaben) | |
| EDETA BD (Disodium EDTA) | 0.10 |
| D-Panthenol | 0.50 |
| Propylene Glycol | 5.00 |
| Kojic Acid | 1.00 |
| Naloxone | 1.00 |
| Water deionized | Ad 100 |

### EXAMPLE 7

Skin Lightening Gel

| Ingredients | % (w/w) |
|---|---|
| Pemulen TR-1 (Acrylate/C10-30 Alkyl | 0.80 |
| Acrylate Crosspolymer) | |
| Biotin | 0.01 |
| EDETA BD (Disodium EDTA) | 0.10 |
| D-Panthenol | 0.20 |
| Hyasol BT (Sodium Hyaluronate) | 1.00 |
| Euxyl K 400 (Methyldibromo Glutaronitrile | 0.20 |
| & Phenoxyethanol) | |
| NaOH (30%) | 1.00 |
| Propylene Glycol | 5.00 |
| Epigallocatechin Gallate | 0.50 |
| Genistein | 0.10 |
| Niacinamide | 0.50 |
| Emblica (Phyllanthus Emblica fruit extract) | 0.50 |
| Hydroquinone | 0.20 |
| Naloxone | 2.00 |
| Citric Acid (10%) | q.s. |
| Water deionized | ad 100 |

### EXAMPLE 8

Skin Lightening Lotion

| Ingredients | % (w/w) |
|---|---|
| Propylene Glycol | 5.00 |
| Naloxone | 1.00 |
| D-Panthenol | 0.50 |
| Sodium PCA | 0.25 |
| Ethanol | 10.00 |
| Citric Acid (10%) | q.s. |
| Water deionized | Ad 100 |

### EXAMPLE 9

Skin lightening cream with UV protection (indicative SPF: 8, O/W)

| Ingredients | % (w/w) |
|---|---|
| PARSOL SLX (Dimethico Diethylbenzalmalonate) | 8.00 |
| Uvinul Titanium Dioxide (Titanium Dioxide) | 2.00 |
| Tegosoft TN (C12-15 Alkyl Benzoate) | 5.00 |
| Silicone 2503 Cosmetic Wax (Stearyl Dimethicone) | 2.00 |
| Cetyl Alcohol | 1.00 |
| Butylated Hydroxytoluene (BHT) | 0.05 |
| Estol GMM 3650 (Glyceryl Myristate) | 4.00 |
| Edeta BD (Disodium EDTA) | 0.10 |
| Phenonip (Phenoxyethanol & Methylparaben | 0.60 |
| & Ethylparaben & Propylparaben & Butylparaben) | |
| AMPHISOL K (Potassium Cetyl Phosphate) | 2.00 |
| Carbopol 980 (Carbomer) | 10.00 |
| Naloxone | 1.00 |
| Propylene Glycol | 5.00 |
| KOH sol. 10% | 0.50 |
| Water deionized | ad 100 |

### EXAMPLE 10

Skin Lightening Gel

| Ingredients | % (w/w) |
|---|---|
| Pemulen TR-1 (Acrylate/C10-30 Alkyl | 0.80 |
| Acrylate Crosspolymer) | |
| EDETA BD (Disodium EDTA) | 0.10 |
| D-Panthenol | 0.10 |
| Hyasol BT (Sodium Hyaluronate) | 1.00 |
| Euxyl K 400 (Methyldibromo Glutaronitrile | 0.20 |
| & Phenoxyethanol) | |
| NaOH (30%) | 1.00 |
| Propylene Glycol | 5.00 |
| Melawhite (Water and Leucocyte extract) | 1.00 |
| Kojic Acid | 0.50 |
| Niacinamide | 0.50 |
| Hydroquinone | 0.50 |
| Naloxone | 1.00 |
| Citric Acid (10%) | q.s. |
| Water deionized | Ad 100 |

### EXAMPLE 11

Skin Lightening Gel

| Ingredients | % (w/w) |
|---|---|
| Pemulen TR-1 (Acrylate/C10-30 Alkyl | 0.80 |
| Acrylate Crosspolymer) | |
| Biotin | 0.01 |
| EDETA BD (Disodium EDTA) | 0.10 |
| D-Panthenol | 0.10 |
| Hyasol BT (Sodium Hyaluronate) | 1.00 |
| Euxyl K 400 (Methyldibromo Glutaronitrile | 0.20 |
| & Phenoxyethanol) | |
| NaOH (30%) | 1.00 |
| Propylene Glycol | 5.00 |
| Melfade (Water and Glycerin and Bearberry extract) | 1.00 |
| Kojic Acid | 0.50 |
| Niacinamide | 0.50 |
| Naloxone | 1.00 |
| Citric Acid (10%) | q.s. |
| Water deionized | Ad 100 |

### EXAMPLE 12

Skin Lightening Gel

| Ingredients | % (w/w) |
|---|---|
| Pemulen TR-1 (Acrylate/C10-30 Alkyl | 0.80 |
| Acrylate Crosspolymer) | |
| EDETA BD (Disodium EDTA) | 0.10 |
| D-Panthenol | 0.10 |
| Hyasol BT (Sodium Hyaluronate) | 1.00 |
| Euxyl K 400 (Methyldibromo Glutaronitrile | 0.20 |
| & Phenoxyethanol) | |
| NaOH (30%) | 1.00 |
| Propylene Glycol | 10.00 |
| Licorice extract | 0.50 |
| Mulberry extract | 0.50 |
| Kojic Acid | 0.50 |
| Niacinamide | 0.50 |
| Naloxone | 0.50 |
| Citric Acid (10%) | q.s. |
| Water deionized | ad 100 |

### EXAMPLE 13

Skin Lightening Gel

| Ingredients | % (w/w) |
|---|---|
| Carbopol ETD 2020 (Carbomer) | 0.80 |
| Panthenol | 0.50 |
| Niacinamide | 0.10 |
| NaOH (30%) | 0.50 |
| Ethanol | 35.00 |
| Propylene Glycol | 8.00 |
| Naloxone | 2.00 |
| Water deionized | ad 100 |

### EXAMPLE 14

Skin lightening cream with UV protection (indicative SPF: 8, O/W)

| Ingredients | % (w/w) |
|---|---|
| Parsol 1789 (Butyl Methoxydibenzoylmethane | 1.50 |
| Uvinul Titanium Dioxide (Titanium Dioxide) | 3.00 |
| Parsol MCX (Ethyl Hexylmethoxycinnamate) | 4.00 |
| Tegosoft TN (C12-15 Alkyl Benzoate) | 8.00 |
| Silicone 2503 Cosmetic Wax (Stearyl Dimethicone) | 2.00 |
| Cetyl Alcohol | 1.00 |
| Butylated Hydroxytoluene (BHT) | 0.05 |
| Estol GMM 3650 (Glyceryl Myristate) | 4.00 |
| Edeta BD (Disodium EDTA) | 0.10 |
| Phenonip (Phenoxyethanol & Methylparaben | 0.60 |
| & Ethylparaben & Propylparaben & Butylparaben) | |
| AMPHISOL K (Potassium Cetyl Phosphate) | 2.00 |
| Carbopol 980 (Carbomer) | 10.00 |
| Naloxone | 2.00 |
| Propylene Glycol | 5.00 |
| KOH sol. 10% | 0.50 |
| Water deionized | ad 100 |

### EXAMPLE 15

Skin lightening cream with UV protection (indicative SPF: 10, O/W)

| Ingredients | % (w/w) |
|---|---|
| PARSOL SLX (Polysilicone 15) | 6.00 |
| PARSOL 1789 (Butyl Methoxydibenzoylmethane) | 2.00 |
| Parsol MCX (Ethyl Hexylmethoxycinnamate) | 4.00 |
| Softisan 100 (Hydrogenated Coco-Glycerides) | 2.00 |
| Glyceryl Myristate | 4.00 |
| Myritol 318 (Caprylic/Capric Triglyceride) | 7.00 |
| Cosmacol ESI (Tridecyl Salicylate) | 8.00 |
| VITAMIN E ACETATE (Tocopheryl Acetate) | 0.50 |
| Phenonip (Phenoxyethanol & Methylparaben | 0.80 |
| & Ethylparaben & Propylparaben & Butylparaben | |
| AMPHISOL K (Potassium Cetyl Phosphate) | 2.00 |
| 1,2-Propylene Glycol (Propylene Glycol) | 5.00 |
| Carbopol ETD 2020 (Acrylate/C10-30 Alkyl | 0.30 |
| Acrylate Crosspolymer) | |
| Edeta BD (Disodium EDTA) | 0.10 |
| KOH 10% sol.(Potassium Hydroxide) | 1.60 |
| STAY-C 50 (Sodium Ascorbyl Phosphate) | 1.00 |
| Naloxone | 2.00 |
| Retinyl Palmitate | 0.50 |
| VITAMIN E (Tocopherol) | 0.10 |
| Water deionized | ad 100 |

### EXAMPLE 16

Skin lightening liquid soap

| Ingredients | % (w/w) |
|---|---|
| Texapon NSO (Sodium Laureth Sulfate) | 40.00 |
| Tego Betain L7 (Cocamidopropyl Betaine) | 10.00 |
| Lamepon S (Potassium Cocoyl Hydrolysed Collagen) | 5.00 |
| Plantaren 1200 (Lauryl Glucoside) | 5.00 |
| Cetiol HE (PEG-7 Glyceryl Cocoate) | 3.00 |
| Preservative | q.s. |
| Polymer JR 400 (Polyquaternium-10) | 0.20 |
| Panthenol 75 L (Panthenol) | 0.40 |
| Naloxone | 1.00 |
| EDETA BD (Disodium EDTA) | 0.10 |
| Vitamine E Acetate (Tocopheryl Acetate) | 0.30 |
| Cremophor RH 40 (PEG-40 Hydrogenated Castor Oil) | 2.00 |
| Sodium Chloride | 1.00 |
| Water deionized | ad 100 |

1. Use of an opioid receptor antagonist for the manufacture of a topical composition for suppression of melanin formation in the human skin.

2. Use of an opioid receptor antagonist for the manufacture of a cosmetic composition for lightening and tanning prevention of the human skin.

3. Use of an opioid receptor antagonist for the manufacture of a pharmaceutical composition for the treatment and prevention of pigmentation disorders.

4. The use as in claim 1 wherein the opioid receptor antagonist is an opioid analogue, a peptide or polypeptide, or a piperidine.

5. The use as in claim 1 wherein the opioid receptor antagonist is an antagonist of μ-opioid receptors.

6. The use as in claim 5 wherein the opioid receptor antagonist is naloxone, naloxonazine, isocembrol, 2′,3′,4′,3,4-pentahydroxychalcone, aureusidin or 2,3-dimethoxy-5-methylhydroquinone, in particular naloxone, isocembrol, 2′,3′,4′₁3,4-pentahydroxychalcone, aureusidin or 2,3-dimethoxy-5-methylhydroquinone.

7. The use as in claim 1 of an opioid receptor antagonist in combination with at least one additional skin lightening agent and/or a UV screening agent.

8. A topical composition for reducing melanin in the human skin, for lightening and tanning prevention of the human skin and/or for the treatment and prevention of pigmentation disorders comprising an opioid receptor antagonist and a conventional carrier.

9. A topical composition comprising an opioid receptor antagonist and at least one additional skin lightening agent and/or a UV screening agent, and a conventional carrier.

10. The composition as in claim 8 wherein the opioid receptor antagonist is an antagonist of μ-opioid receptors.

11. The composition as in claim 10 wherein the opioid receptor antagonist is naloxone, naloxonazine, isocembrol, 2′,3′,4′,3,4-pentahydroxychalcone, aureusidin or 2,3-dimethoxy-5-methylhydroquinone, in particular naloxone, isocembrol, 2′,3′,4′,3,4-pentahydroxychalcone, aureusidin or 2,3-dimethoxy-5-methylhydroquinone.

12. The composition as in claim 8 which is a cream, a gel, an ointment, a lotion, a tincture, a spray, a mousse, a cleansing composition or foam.

13. The composition as in claim 8 wherein the opioid receptor antagonist is present in an amount of from about 0.00001 wt.-% to about 20 wt.-%, preferably from about from about 0.0001 wt.-% to about 10 wt.-%, more preferably from about from about 0.005 wt.-% to about 5 wt.-%.

14. A method of lightening human skin which comprises topically administering an effective amount of an opioid receptor antagonist to the appropriate skin area of a person in need of such treatment.

15. A method of treating or preventing pigmentation disorders which comprises topically administering an effective amount of an opioid receptor antagonist to the appropriate skin area of a person in need of such treatment.

16. The method as in claim 14 wherein from about 0.2 μg to about 200 μg of opioid receptor antagonist are applied per square centimetre of skin per day.

17. The method as in claim 14 wherein the opioid receptor antagonist is an antagonist of μ-opioid receptors.

18. The method as in claim 14 wherein the opioid receptor antagonist is naloxone, naloxonazine, isocembrol, 2′,3′,4′,3,4-pentahydroxychalcone, aureusidin or 2,3-dimethoxy-5-methylhydroquinone, in particular naloxone, isocembrol, 2′,3′,4′,3,4-pentahydroxychalcone, aureusidin or 2,3-dimethoxy-5-methylhydroquinone.

19. The method as in claim 1 wherein at least one additional skin lightening agent and/or a UV screening agent is additionally administered.

20. A method of identifying an agent which can be used to reduce melanin in the human skin which comprises determining the activity of said agent to inhibit an opioid receptor, particularly a μ-opioid receptor.

21. The method as in claim 20 which comprises determining whether the agent causes a substantial reduction of the total intracellular melanin production in human primary melanocytes from skin type IV.

22. The method as in claim 21 wherein a reduction of the total intracellular melanin production of at least 20% is regarded as indicative of a usefulness of the agent for inhibiting melanin formation and for skin lightening when applied as a topical composition.

23. The invention as particularly described hereinbefore, especially with reference to the Examples.
